(19) United States
(12) Patent Application Publication
(10) Pub. No.: US 2024/0221946 A1
(43) Pub. Date: Jul. 4, 2024

(54) LEARNING METHOD AND DEVICE FOR ALZHEIMER PREDICTION MODEL BASED ON DOMAIN ADAPTATION

(71) Applicant: AJOU UNIVERSITY INDUSTRY-ACADEMIC COOPERATION FOUNDATION, Suwon-si (KR)

(72) Inventors: Hyunjung Shin, Suwon-si (KR); Chang Hyung Hong, Seongnam-si (KR); Sang Joon Son, Seoul (KR); Hyun Woong Roh, Suwon-si (KR); Sunghong Park, Suwon-si (KR)

(73) Assignee: AJOU UNIVERSITY INDUSTRY-ACADEMIC COOPERATION FOUNDATION, Suwon-si (KR)

(21) Appl. No.: 18/401,741

(22) Filed: Jan. 2, 2024

(30) Foreign Application Priority Data
Dec. 30, 2022 (KR) 10-2022-0190673
Dec. 29, 2023 (KR) 10-2023-0196220

(51) Int. Cl. G16H 50/20; A61B 5/00

(52) U.S. Cl. CPC G16H 50/20; A61B 5/4088; A61B 5/7275

(57) ABSTRACT

Disclosed is a learning method and device for alzheimer prediction model based on domain adaptation performed by at least one processor including extracting a point related to an object in a learning image from the learning image for a 3D reconstruction model, obtaining a gradient map including surrounding context information in three dimensions of the point from a 3D model of the object, determining a weight of the point based on the learning image and the gradient map, and learning the 3D reconstruction model by using the weight such that the 3D model of the object is output from the 3D reconstruction model into which the learning image is input.

### CROSS-REFERENCE TO RELATED APPLICATIONS

This application claims priority under 35 U.S.C. § 119 to Korean Patent Application No. 10-2022-0190673 filed on Dec. 30, 2022 and Korean Patent Application No. 10-2023-0196220 filed on Dec. 29, 2023, in the Korean Intellectual Property Office, the disclosures of which are incorporated by reference herein in their entireties.

### BACKGROUND

Embodiments of the present disclosure described herein relate to a learning device for alzheimer prediction model based on domain adaptation, and a method thereof.

Conventionally, Alzheimer's disease (AD) is the most common form of dementia affecting older adults. Due to the extension of life expectancy, the number of Alzheimer's disease patients continues to increase, and the global Alzheimer's disease population is expected to triple from approximately 50 million in 2015 to 131.5 million in 2050. As such, AD is emerging as a serious problem, but the cause is unclear and there is no cure. Therefore, it is important to detect AD early, predict the potential risk of disease progression, and find appropriate prevention strategies. According to clinical symptoms accompanying mild memory loss or other cognitive impairment, Mild Cognitive Impairment (MCI) is a prodromal stage of AD accompanied by symptoms such as long-term memory loss, language impairment, disorientation, and personality changes. It is considered. Previous studies have shown that approximately 12% of subjects suffering from MCI progress to AD within 4 years after first symptoms.

### SUMMARY

Embodiments of the present disclosure provide a learning device for 3D model reconstruction that enables compatibility between 3D data and an existing 2D model, and a method thereof.

According to an embodiment of the present disclosure, learning method for alzheimer prediction model based on domain adaptation includes acquiring a target dataset of a target domain associated with a first brain, acquiring a source dataset of a source domain associated with a second brain—the source dataset includes a label indicating diagnostic information about the second brain —, operating domain adaptation on the target dataset based on the source dataset to obtain a transformed target dataset and transformed target data. It includes the step of training a machine learning model using the set and source dataset as learning data, and the machine learning model is data containing information related to the possibility of conversion from mild cognitive impairment to Alzheimer's disease as data related to the brain is input. is learned to be output, and the objective function of the machine learning model can be constructed based on the difference between the transformed target dataset and the source dataset.

According to one embodiment, the objective function is based on an adaptation matrix, the adaptation matrix is configured to reduce the difference between the first mean vector obtained from the target dataset and the second mean vector obtained from the source dataset, the first average vector may correspond to the center of the data distribution according to the label of the target dataset, and the second average vector may correspond to the center of the data distribution according to the label of the source dataset.

According to one embodiment, the first average vector may be obtained from a target data matrix corresponding to the target dataset, and the second average vector may be obtained from a source data matrix corresponding to the source dataset.

According to one embodiment, the objective function is constructed based on [Equation 1] below,

$$‖ {X}^{-} \_TA - {X}^{-} \_S ‖ \_ 2 \hat{} 2 \qquad [ Equation 1 ]$$

In Equation 1, A may correspond to the adaptation matrix, X⁻_T may correspond to the first average vector, and X⁻_S may correspond to the second average vector.

According to one embodiment, the objective function is based on an adaptation matrix, and the adaptation matrix reduces the difference between the first correlation matrix obtained from the target dataset and the second correlation matrix obtained from the source dataset. Additional steps may be included.

According to one embodiment, the first correlation matrix includes information associated with correlations between regions of interest (ROIs) associated with the first brain, and the second correlation matrix includes information associated with correlations between regions of interest (ROIs) associated with the second brain. May contain information related to correlation.

According to one embodiment, the objective function is constructed based on [Equation 2] below,

$$‖ A \hat{} TC\_TA - C\_S ‖ \_ 2 \hat{} 2 \qquad [ Equation 2 ]$$

In Equation 2, A may correspond to the adaptation matrix, C_T may correspond to the first correlation matrix, and C_S may correspond to the second correlation matrix.

According to another embodiment of the present disclosure, a computer program recorded on a computer-readable recording medium may be provided to execute a domain adaptation-based Alzheimer's prediction model learning method.

### BRIEF DESCRIPTION OF THE FIGURES

The above and other objects and features of the present disclosure will become apparent by describing in detail embodiments thereof with reference to the accompanying drawings.

FIGS. 1A to 1C are schematic diagrams showing a method for learning a domain adaptation-based Alzheimer's prediction model according to an embodiment of the present disclosure.

FIG. 2 is a block diagram showing the internal configuration of an information processing system that performs a domain adaptation-based Alzheimer's prediction model learning method according to an embodiment of the present disclosure.

FIG. 3 is a flowchart of a method for learning a domain adaptation-based Alzheimer's prediction model according to an embodiment of the present disclosure.

### DETAILED DESCRIPTION

Hereinafter, details for implementing the present disclosure will be described in detail with reference to the accompanying drawings. However, in the following description, when there is a risk of unnecessarily obscuring the gist of the present disclosure, detailed descriptions of well-known functions or configurations will be omitted.

In the accompanying drawings, identical or corresponding components are assigned the same reference numerals. Moreover, in the description of embodiments below, descriptions of the same or corresponding components may be omitted to avoid redundancy. However, even though descriptions regarding components are omitted, it is not intended that such components are not included in any embodiment.

The above and other aspects, features and advantages of the present disclosure will become apparent from embodiments to be described in conjunction with the accompanying drawings. However, the present disclosure may be embodied in various different forms, and should not be construed as being limited only to the illustrated embodiments. Rather, these embodiments are provided as examples such that the present disclosure will be thorough and complete, and will fully convey the scope of the present disclosure to those skilled in the art.

Terms used in this specification will be briefly described, and the disclosed embodiments will be described in detail. Although certain general terms widely used in this specification are selected to describe embodiments in consideration of the functions thereof, these general terms may vary according to intentions of one of ordinary skill in the art, case precedents, the advent of new technologies, and the like. Terms arbitrarily selected by the applicant of the embodiments may also be used in a specific case. In this case, their meanings are given in the detailed description of the present disclosure. Hence, these terms used in the present disclosure may be defined based on their meanings and the contents of the present disclosure, not by simply stating the terms.

Expressions in the singular used in this specification include a plurality of expressions unless interpreted otherwise in context. A plurality of expressions includes expressions in the singular unless the context clearly dictates that the expression is plural. It will be understood that the terms “comprises,” “comprising,” “includes” and/or “including,” when used herein, specify the presence of stated elements and/or components, but do not preclude the presence or addition of one or more other elements and/or components.

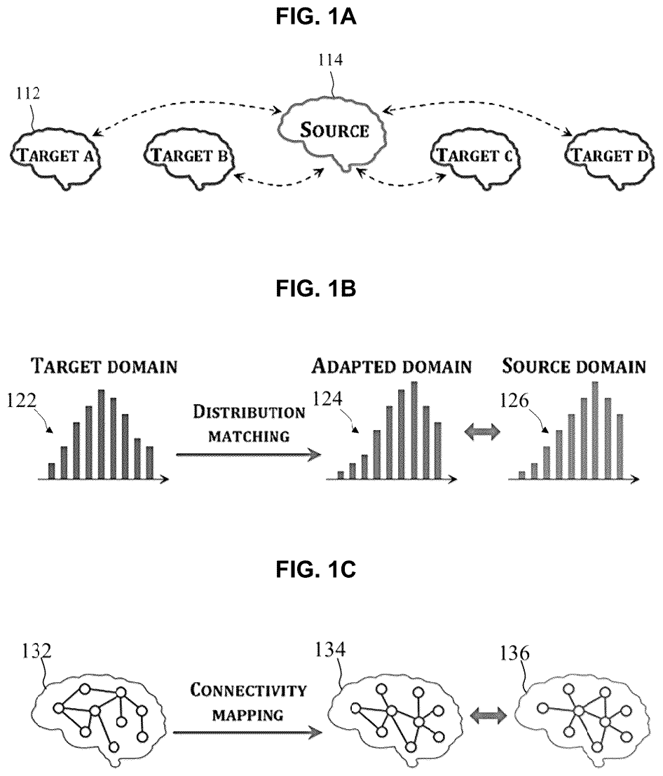

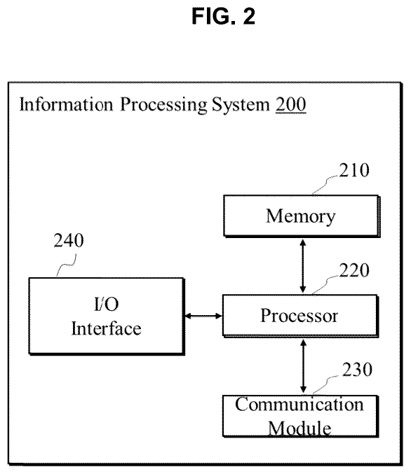

FIGS. 1A to 1C are schematic diagrams showing a domain adaptation-based Alzheimer's prediction model learning method (hereinafter referred to as “method”) according to an embodiment of the present disclosure. Specifically, FIG. 1A is a schematic diagram showing the entire process of the method. Next, FIGS. 1B and 1C are schematic diagrams showing some processes of the method, respectively. Meanwhile, the method in the present disclosure may be performed by at least one processor of an information processing system (described later in FIG. 2).

Referring to FIG. 1A, the processor may perform the method by operating domain adaptation between one target domain 112 and the source domain 114. Furthermore, the processor may perform the method by operating domain adaptation with each of the plurality of target domains 112 (here, Targets A to D). Specifically, the processor may perform domain adaptation by transforming the target dataset obtained from the target domain 112 to be similar to the source dataset obtained from the source domain 114. That is, the processor can perform domain adaptation by transforming only the target dataset without transforming the source dataset. With this configuration, data loss that occurs during the process of converting a source dataset with relatively excellent data quality and quantity can be prevented. For example, the source dataset may represent a dataset obtained from a database of a large hospital, and the target dataset may represent a dataset obtained from a database of a small hospital.

Referring to FIG. 1B, the processor may perform domain adaptation for the target dataset by transforming the data distribution 122 of the target dataset to be similar to the data distribution 126 of the source dataset. Here, ‘data distribution’ may mean data distribution in the feature space of each domain. Specifically, the processor may obtain a target data matrix X_T representing the data distribution 122 of the target dataset, and obtain a first average vector Similarly, the processor may obtain a source data matrix X_S representing the data distribution 126 of the source data set, and obtain a second average vector Here, the first average vector may correspond to the center of the data distribution 122 of the target dataset. Likewise, the second mean vector may correspond to the center of the data distribution 126 of the source dataset.

The processor may use an adaptation matrix to reduce the difference between the center of the data distribution 122 of the target dataset and the center of the data distribution 126 of the source dataset. Specifically, the processor may narrow (or minimize) the distance between the data distribution center of the target data set and the distribution center of the source data set according to Equation 1 below based on the adaptation matrix A. Accordingly, the processor may obtain a modified target dataset having a data distribution 124 whose data distribution center is similar to the source dataset.

$$‖ {X}^{-} \_TA - {X}^{-} \_S ‖ \_ 2 \hat{} 2 \qquad [ Equation 1 ]$$

Additionally or alternatively, referring to FIG. 1C, the processor may transform the inter-data correlation 132 of the target dataset to be similar to the inter-data correlation 136 of the source dataset. Domain adaptation can also be performed. Here, ‘correlation between data’ may mean the positional relationship in the brain between one piece of data and another piece of data in the feature space of each domain. More specifically, when the first region of interest and the second region of interest extracted from one brain image are located adjacent to (or adjacent to) the image, the first data and the second region of interest corresponding to the first region of interest The second data corresponding to may indicate a relationship in the feature space. Specifically, the processor may obtain the first correlation matrix C_T from the target dataset. Similarly, the processor may obtain a second correlation matrix C_S from the source dataset.

$$‖ A \hat{} TC\_TA - C\_S ‖ \_ 2 \hat{} 2 \qquad [ Equation 2 ]$$

The processor may reduce the difference between the correlation of the target dataset and the correlation of the source dataset based on the adaptation matrix A, the first correlation matrix C_T, and the second correlation matrix C_S. That is, the processor may map the correlation of the source dataset to the correlation of the target dataset based on the adaptation matrix A, the first correlation matrix C_T, and the second correlation matrix C_S according to Equation 2. Accordingly, the process can obtain a transformed target dataset having a correlation 134 between transformed data.

FIG. 2 is a block diagram showing the internal configuration of an information processing system 200 that performs a domain adaptation-based Alzheimer's prediction model learning method according to an embodiment of the present disclosure. The information processing system 200 may include a memory 210, a processor 220, a communication module 230, and an input/output interface 240. As shown in FIG. 2, the information processing system 200 may be configured to communicate information and/or data through a network using each communication module 230.

Memory 210 may include any non-transitory computer-readable recording medium. According to one embodiment, the memory 210 is a non-permanent mass storage device such as random access memory (RAM), read only memory (ROM), disk drive, solid state drive (SSD), flash memory, etc. mass storage device). As another example, non-perishable mass storage devices such as ROM, SSD, flash memory, disk drive, etc. may be included in the information processing system 200 as a separate persistent storage device that is distinct from memory. Additionally, the memory 210 may store an operating system and at least one program code (eg, a code for controlling the information processing system 200 installed and driven in the information processing system 200).

These software components may be loaded from a computer-readable recording medium separate from the memory 210. Recording media readable by such a separate computer may include recording media directly connectable to the information processing system 200, for example, floppy drives, disks, tapes, DVD/CD-ROM drives, memory cards, etc. It may include a recording medium that can be read by a computer. As another example, software components may be loaded into the memory 210 through the communication module 230 rather than a computer-readable recording medium. For example, at least one program may be loaded into the memory 210 based on a computer program installed by files provided over a network by developers or a file distribution system that distributes application installation files.

The processor 220 may be configured to process instructions of a computer program by operating basic arithmetic, logic, and input/output operations. Commands may be provided to the processor 220 by the memory 210 or the communication module 230. For example, processor 220 may be configured to execute received instructions according to program code stored in a recording device such as memory 210.

The communication module 230 may provide a configuration or function for the information processing system 200 to communicate with another system (for example, a separate cloud system, etc.) through a network. For example, a control signal or command provided under the control of the processor 220 of the information processing system 200 may be received by another system through the communication module 230 and a network.

The input/output interface 240 may be connected to the information processing system 200 or may be a means for interfacing with a device (not shown) for input or output that the information processing system 200 may include. In FIG. 2, the input/output interface 240 is shown as an element configured separately from the processor 220, but the present invention is not limited thereto, and the input/output interface 240 may be included in the processor 220.

Information processing system 200 may include more components than those in FIG. 2. However, there is no need to clearly show most prior art components.

According to one embodiment, the processor 220 may be configured to operate a program for learning and inference of a neural network. At this time, code related to the program may be loaded into the memory 210. While the program is operating, the processor 220 receives information and/or data provided from an input/output device (not shown) through the input/output interface 240 or receives information and/or data from another system through the communication module 230. The received information and/or data can be processed and stored in the memory 210. Additionally, such information and/or data may be provided to other systems through the communication module 230.

Processor 220 may be configured to manage, process, and/or store information and/or data received from a plurality of other systems. According to one embodiment, the processor 220 may store, process, and transmit images received from other systems, features extracted from the images, etc. Additionally or alternatively, the processor 220 may be configured to store and/or update a program for executing an algorithm used for learning and inference of an artificial neural network from a separate cloud system, database, etc. connected to the network.

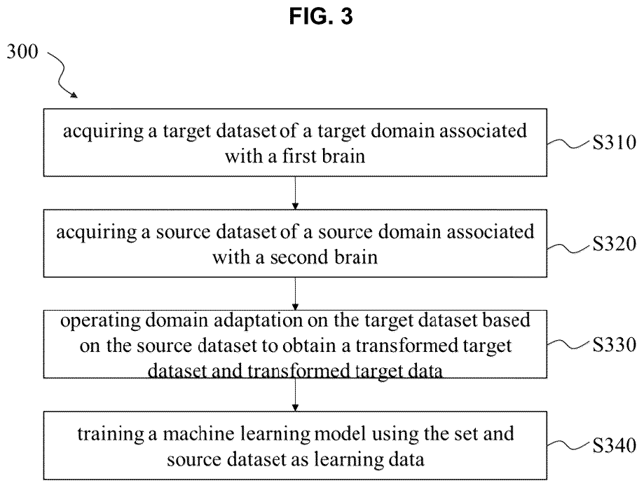

FIG. 3 is a flowchart of a domain adaptation-based Alzheimer's prediction model learning method 300 according to an embodiment of the present disclosure. Method 300 may be performed by at least one processor (eg, processor 220) of an information processing system.

As shown, the method 300, according to an embodiment of the present disclosure, the domain adaptation-based Alzheimer's prediction model learning method may be initiated with a step (S310) of acquiring a target dataset of a target domain associated with the first brain. there is. Then, the processor may obtain the source dataset of the source domain associated with the second brain (S320). In this case, the source dataset may include a label indicating diagnostic information about the second brain. Meanwhile, in FIG. 3, the step of acquiring the target dataset of the target domain associated with the first brain (S310) is shown to be performed before the acquisition of the source dataset of the source domain associated with the second brain (S320), but is not limited to this. No. For example, the two steps (S310 and S320) may be performed in reverse order or in parallel (i.e., simultaneously).

The processor may obtain a transformed target dataset by operating domain adaptation on the target dataset based on the source dataset (S330). Then, the processor can train a machine learning model using the transformed target dataset and source dataset as learning data (S340). In this case, the machine learning model can be trained to output data containing information related to the possibility of conversion from mild cognitive impairment to Alzheimer's disease as brain-related data is input. Additionally or alternatively, the objective function of the machine learning model may be constructed based on the differences between the transformed target dataset and the source dataset.

According to one embodiment, the objective function is based on an adaptation matrix, the adaptation matrix is configured to reduce the difference between the first mean vector obtained from the target dataset and the second mean vector obtained from the source dataset, The first average vector may correspond to the center of the data distribution according to the label of the target dataset, and the second average vector may correspond to the center of the data distribution according to the label of the source dataset.

According to one embodiment, the first average vector may be obtained from a target data matrix corresponding to the target dataset, and the second average vector may be obtained from a source data matrix corresponding to the source dataset.

According to one embodiment, the objective function is constructed based on [Equation 1] below,

$$‖ {X}^{-} \_TA - {X}^{-} \_S ‖ \_ 2 \hat{} 2 \qquad [ Equation 1 ]$$

In Equation 1, A may correspond to the adaptation matrix, X⁻_T may correspond to the first average vector, and X⁻_S may correspond to the second average vector.

According to one embodiment, the objective function is based on an adaptation matrix, and the adaptation matrix reduces the difference between the first correlation matrix obtained from the target dataset and the second correlation matrix obtained from the source dataset. Additional steps may be included.

According to one embodiment, the first correlation matrix includes information associated with correlations between regions of interest (ROIs) associated with the first brain, and the second correlation matrix includes information associated with correlations between regions of interest (ROIs) associated with the second brain. May contain information related to correlation.

According to one embodiment, the objective function is constructed based on [Equation 2] below,

$$‖ A \hat{} TC\_TA - C\_S ‖ \_ 2 \hat{} 2 \qquad [ Equation 2 ]$$

In Equation 2, A may correspond to the adaptation matrix, C_T may correspond to the first correlation matrix, and C_S may correspond to the second correlation matrix. The previous description of the present disclosure is provided to enable those skilled in the art to make or use the present disclosure. Various modifications of the present disclosure will be easily apparent to those skilled in the art, and the generic principles defined herein may be applied to various modifications without departing from the spirit or scope of the present disclosure. Accordingly, the present disclosure is not intended to be limited to the examples set forth herein, but is to be accorded the widest scope consistent with the principles and novel features disclosed herein. Although the present disclosure has been described herein in connection with some embodiments, it should be understood that various modifications and changes may be made without departing from the scope of the present disclosure as understood by those skilled in the art to which the present disclosure pertains. Moreover, such modifications and variations are intended to fall within the scope of claims appended hereto.

While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A learning method and device for alzheimer prediction model based on domain adaptation performed by at least one processor, the method comprising:
acquiring a target dataset of a target domain associated with a first brain,
acquiring a source dataset of a source domain associated with a second brain—the source dataset includes a label indicating diagnostic information about the second brain—,
operating domain adaptation on the target dataset based on the source dataset to obtain a transformed target dataset and transformed target data; and
training a machine learning model using the set and source dataset as learning data,
wherein the machine learning model is data containing information related to the possibility of conversion from mild cognitive impairment to Alzheimer's disease as data related to the brain is input. is learned to be output,
wherein an objective function of the machine learning model can be constructed based on the difference between the transformed target dataset and the source dataset.

2. The method of claim 1,
wherein the objective function is based on an adaptation matrix,
the adaptation matrix is configured to reduce the difference between a first average vector obtained from the target dataset and a second average vector obtained from the source dataset,
the first average vector corresponds to the center of the data distribution according to the label of the target dataset, and
the domain adaptation-based Alzheimer's prediction model learning method performed by at least one processor, wherein the second average vector corresponds to the center of the data distribution according to the label of the source dataset.

3. The method of claim 2,
wherein the first average vector is obtained from a target data matrix corresponding to the target data set, and
the second average vector is obtained from a source data matrix corresponding to the source dataset.

4. The method of claim 3,
wherein the objective function is constructed based on the following [Equation 1],
$$‖ {X}^{-} \_TA - {X}^{-} \_S ‖ \_ 2 \hat{} 2 \qquad [ Equation 1 ]$$
In Equation 1, A corresponds to the adaptation matrix, X⁻_T corresponds to the first average vector, and X⁻_S corresponds to the second average vector.

5. The method of claim 1,
wherein the objective function is based on an adaptation matrix,
the adaptation matrix reduces the difference between a first correlation matrix obtained from the target dataset and a second correlation matrix obtained from the source dataset.

6. The method of claim 1,
wherein the first correlation matrix includes information related to correlations between regions of interest (ROIs) associated with the first brain, and
the second correlation matrix includes information related to correlation between regions of interest associated with the second brain.

7. The method of claim 1,
wherein The objective function is constructed based on the following [Equation 2],
$$‖ A \hat{} TC\_TA - C\_S ‖ \_ 2 \hat{} 2 \qquad [ Equation 2 ]$$
In Equation 2, A corresponds to the adaptation matrix, C_T corresponds to the first correlation matrix, and C_S corresponds to the second correlation matrix.

8. A computer-readable recording medium which records a computer program to perform learning method for alzheimer prediction model based on domain adaptation according to claim 1.
